(19) United States
(12) Patent Application Publication
(10) Pub. No.: US 2011/0230399 A1
(43) Pub. Date: Sep. 22, 2011

(54) Methods for Treating Obesity Related Disease

(76) Inventor: Richard L. Bowen, Raleigh, NC (US)

(21) Appl. No.: 13/130,885

(22) PCT Filed: Nov. 24, 2009
(86) PCT No.: PCT/US09/65643

§ 371 Date: May 24, 2011

Related U.S. Application Data
(60) Provisional application No. 61/117,737, filed on Nov. 25, 2008.

(51) Int. Cl. A61K 38/17; A61P 3/00; A61P 3/04

(52) U.S. Cl. 514/4.9; 514/5.2

(57) ABSTRACT

The present invention relates to methods and products for the treatment of obesity. The invention discloses a method of treating obesity related disease in a mammal in need of such treatment comprising administering to the mammal a therapeutically effective amount of a composition which causes the continued down regulation of the NPY-1 or NPY-5 receptor.

This application claims priority of U.S. provisional application No. 61/117,737 filed on Nov. 25, 2008 and is included herein by reference in its entirety.

### COPYRIGHT NOTICE

A portion of the disclosure of this patent contains material that is subject to copyright protection. The copyright owner has no objection to the reproduction by anyone of the patent document or the patent disclosure as it appears in the Patent and Trademark Office patent files or records, but otherwise reserves all copyright rights whatsoever.

### BACKGROUND OF THE INVENTION

1. Field of the Invention

The present invention relates to a method for slowing, preventing, delaying, or treating excessive appetite, excessive energy (food) intake, excessive energy storage, and inadequate energy expenditure or slowing, preventing, delaying, or treating a disease or condition associated with excessive appetite, excessive energy (food) intake, excessive energy storage, and inadequate energy expenditure (collectively an eating related condition or disease). More particularly, the present invention relates to a method for slowing, preventing, delaying, decreasing eating or treating excessive appetite (desire for food), excessive energy (food) intake, excessive energy storage, and inadequate energy expenditure or slowing, preventing, delaying, or treating a disease or condition associated with excessive appetite, excessive energy (food) intake, excessive energy storage, and inadequate energy expenditure by administering a therapeutically effective amount of at least one physiological agent that activates or causes the activation of and subsequent down regulation of one or more neuropeptide-Y receptors.

2. Description of Related Art

Neuropeptide Y is a 36 amino acid peptide that is widely distributed in the central and peripheral nervous system. Most of the NPY system responsible for regulating food intake is thought to be located in the hypothalamus. This peptide mediates a number of physiological effects through its various receptor subtypes. Studies in animals and humans have shown that neuropeptide Y is a powerful stimulant to food intake (Clark et al. 1984; Levine & Morley 1984; Stanley & Leibowitz 1984), and also has been implicated to decrease thermogenesis. Mashiko S Endocrinology. 2003 May; 144(5):1793-801. It is believed that these effects are mediated through the Y1 and Y5 receptor subtypes, Beck, B Neuropeptide Y in normal eating and in genetic and dietary-induced obesity. Philos Trans R Soc Lond B Biol Sci. 2006 Jul. 29; 361(1471):1159-85.

Antagonists of the NPY receptors have represented an approach to the treatment of eating disorders such as obesity and hyperphagia. For example, in U.S. Pat. No. 7,265,125 issued Sep. 4, 2007 to Breu, et al, there is described a number of quinoline and quinazoline derivatives which are antagonists of the NPY receptors and are described as useful in the treatment of eating disorders and obesity. Also, in WO 2003/009845 to Stamford et al., there are disclosed a number of Y-5 antagonists for the treatment of obesity. Likewise in Medicinal strategies in the treatment of obesity, Bray, et al. Nature, 2000 Apr. 6; 404 (6778):674-5 there is a review of a number of neuropeptides shown to affect food intake. It states that Neuropeptide Y is among the most potent stimulators of feeding and its synthesis and release is modulated by insulin, leptin and starvation. Antagonists to either the Y-5 or the Y-1 subtype receptors are being explored as potential agents for the treatment of obesity. The article also concludes that redundant systems may limit this method of treatment for obesity.

Compounds which increase the release of NPY, Chen H Regulation of hypothalamic NPY by diet and smoking Peptides. 2007 February; 28(2):384-9. Epub 2007 Jan. 17, are known and can result in increased down regulation of NPY receptors when administered. These compounds have also been reported or suggested to increase appetite. It is logical to conclude from this data that the same would be true for compositions that block the degradation or reuptake of NPY.

NPY13-36 does not bind to Y1 receptors, but it is a full agonist at the Y2 receptor. It does not stimulate food intake but inhibits NPY release from hypothalamic slices in vitro (King et al. 1999, 2000).

All NPY receptors including NPY-1 and NPY-5 are members of the 7-transmembrane family of receptors called the g-protein coupled receptors (GPCR). GPCR's are cell surface receptors that signal in the presence of a ligand either naturally occurring or man-made. Antagonists to these receptors are known to block the activation of the receptor when bound to the receptor. As part of their biological pathway, GPCR's normally internalize after signaling for a period of time before returning to the surface of the cell. Internalization occurs after receptor phosphorilization. It is known that blocking the phosphorilization step in a GPCR can lead to a slowing of the internalization process and decreased signaling. For example, it is known that certain kinases are involved in the internalization process and, therefore, interfering with the kinase pathway can modulate the internalization process. In U.S. Pat. No. 6,833,436 modulators of a kinase are taught which can affect the internalization properties of a GPCR. The NPY1 and the NPY5 receptors are well known, for example, as taught in Leibowitz, S. F. & Alexander, J. T. 1991 Analysis of neuropeptide Y-induced feeding—dissociation of Y1-receptor and Y2-receptor effects on natural meal patterns, Peptides 12, 1251-1260. Mullins, D., Kirby, D., Hwa, J., Guzzi, M., Rivier, J. & Parker, E. 2001 Identification of potent and selective neuropeptide YY1 receptor agonists with orexigenic activity in vivo. Mol. Pharmacol. 60, 534-540, Balasubramaniam, A. 1997 Neuropeptide Y family of hormones: receptor subtypes and antagonists. Peptides 18, 445-457, and Blomquist, A. G. & Herzog, H.1997Y-receptor subtypes—Trends Neurosci. 20, 294-298.

Accordingly, in view of the above it would be useful if there were other peptide mediated systems for the treatment of an eating related condition or disease that overcame the problems associated with current treatments. It would be useful, as well, if there were an NPY mediated treatment for an eating related condition or disease state.

### BRIEF SUMMARY OF THE INVENTION

The stimulation of the NPY receptors by any of a number of known means increases appetite. However, it has been discovered in the present invention that if the receptors in a mammal are over stimulated to the point that they are essentially maximally down regulated, that continued over stimulation once down regulation of the receptors occurs, leads to eventual appetite suppression. Accordingly, stimulating the NPY receptor to the point of continuous down regulation can be used to treat an eating related condition or disease.

In an embodiment of the invention there is disclosed a method of treating an eating related condition or disease in a mammal in need of such treatment comprising administering to the mammal a therapeutically effective amount of a composition which causes the continued down regulation of the NPY-1 or NPY-5 receptor.

In yet another embodiment, there is disclosed method for treating an eating related disease or condition in a human in need of such treatment comprising:
a) administering to the human a therapeutically effective amount of a pharmaceutically acceptable composition from the group comprising NPY, an NPY-1 or NPY-5 agonist, an NPY-1 or NPY-5 DAC and a NPY production stimulant;
b) continually administering the composition from a) while monitoring the effect of the composition and observing when appetite enhancement ends; and
c) continuing to administer the composition past the end of appetite enhancement until a desired eating related disease or condition effect is achieved.

### BRIEF DESCRIPTION OF THE DRAWINGS

FIG. 1a is a flow diagram of the NPY regulation of food intake and the known relationship to appetite.

FIG. 1b is a flow diagram of the effect of the present invention on down regulation of receptors and the relationship to appetite.

### DETAILED DESCRIPTION OF THE INVENTION

It has been discovered that stimulating the NPY-1 and 5 receptors to the point of down regulation and then, thereafter, continuously stimulating them such that they remain continuously down regulated, causes a decrease in appetite after the initial appetite stimulation effect. The decrease in appetite thus causes a decrease in food intake and a decrease in weight of the mammal and a positive effect on any an eating related condition or disease connected to that mammal's weight. This affect can be continuous for as long as the medication is given and in some cases can be indefinitely given if desired.

While this invention is susceptible of embodiment in many different forms, there is shown in the one or more drawings and will herein be described in detail specific embodiments, with the understanding that the present disclosure of such embodiments is to be considered as an example of the principles and not intended to limit the invention to the specific embodiments shown and described. In the description below, like reference numerals are used to describe the same, similar or corresponding parts in the several views of the drawings if any. This detailed description defines the meaning of the terms used herein and specifically describes embodiments in order for those skilled in the art to practice the invention.

The terms “a” or “an”, as used herein, are defined as one or more than one. The term “plurality”, as used herein, is defined as two or more than two. The term “another”, as used herein, is defined as at least a second or more. The terms “including” and/or “having”, as used herein, are defined as comprising (i.e., open language). The term “coupled”, as used herein, is defined as connected, although not necessarily directly, and not necessarily mechanically.

Reference throughout this document to “one embodiment”, “certain embodiments”, and “an embodiment” or similar terms means that a particular feature, structure, or characteristic described in connection with the embodiment is included in at least one embodiment of the present invention. Thus, the appearances of such phrases or in various places throughout this specification are not necessarily all referring to the same embodiment. Furthermore, the particular features, structures, or characteristics may be combined in any suitable manner in one or more embodiments without limitation.

The term “or” as used herein is to be interpreted as an inclusive or meaning any one or any combination. Therefore, “A, B or C” means any of the following: “A; B; C; A and B; A and C; B and C; A, B and C”. An exception to this definition will occur only when a combination of elements, functions, steps or acts are in some way inherently mutually exclusive.

As used herein, the phrase “an eating related condition or disease” refers to conditions and diseases that relate to eating a greater amount than is otherwise healthy or metabolically needed for a given individual. That condition or disease not only refers to obesity or being overweight, but also to the myriad of weight and metabolism related conditions and disease states which can be improved by weight loss, appetite control etc, many of which are described above. Treating such a condition or disease state refers to ameliorating or otherwise improving in a perceptible amount or way, the state of the mammal, such as a human, who is treated for an eating related condition or disease. A clinical improvement in the weight condition (e.g. weight loss) is easily measured by weight, girth or other known measurements, % body fat via DEXA, subcutaneous fat via calipers, and the like. A clinical improvement in a disease state caused by the weight or size of the mammal can be measured not only by the weight loss but in the relationship to improvement obtained in the disease state from such weight loss. For example, loss of weight in an adult onset diabetic can drastically improve and in some cases cure the diabetic condition. Loss of weight can also, for example, decrease the measurable bad cholesterol and thus lead to decrease in cardiovascular disease related thereto. In other words, any condition or disease that improves with the loss of weight in the mammal is considered an eating related condition or disease. Other diseases that would benefit are well known, as described above, but in general any disease being treated by a loss in weight is within the scope of the present invention, such as diabetes, hypertension, cancer, and the like. Improvement includes but is not limited to treating, mitigating, slowing the progression of, or preventing excessive appetite or treating, mitigating, slowing the progression of, or preventing obesity or treating, mitigating, slowing the progression of, or preventing excessive body fat or treating, mitigating, slowing the progression of, or preventing excessive body weight or preventing or slowing proliferation of adipocytes or preventing or slowing lipogenesis or increasing lipolysis or treating, mitigating, slowing the progression of, or preventing excessive adipocyte size or treating, mitigating, slowing the progression of, or preventing excessive body mass or body mass index or treating, mitigating, slowing the progression of, or preventing metabolic syndrome or treating, mitigating, slowing the progression of, or preventing other eating related condition or disease.

As used herein “down regulation” refers to the process of developing a refractory or tolerant state consequent upon repeated administration of a pharmacologically or physiologically active substance to an individual. It is, in general, a process that decreases ligand and receptor interactions or reduces the responsiveness of a cell to a stimulus following first exposure. This process is one that can be affected due to the process of interconnected compositions in the system of a mammal which control the production of compositions that in turn regulate the production of other compositions which turn on or off the particular systems involved in regulatory systems of the body. As an example of down regulation, gonadotropin releasing hormone (GnRH) agonists represent an example of the therapeutic use of G-coupled protein receptor down-regulation. GnRH is a member of the hypothalamic-pituitary-gonadal (HPG) axis. The HPG axis is responsible for the regulation of reproductive function in humans. GnRH is released by the hypothalamus into the hypothalamic-pituitary portal circulation and binds to GnRH receptors in the pituitary. This stimulates gonadotropic cells in the pituitary to release the gonadotropins luteinizing hormone (LH) and follicle stimulating hormone (FSH) into the peripheral circulation. LH and FSH in turn bind to receptors on the gonads and stimulate the production of the sex steroids and inhibin. The elevated levels of circulating sex steroids and inhibin then complete the axis by binding to receptors on the hypothalamus and pituitary and inhibit the release of GnRH, LH, and FSH. Another example of down regulation is discussed in “the regulation of G Protein-coupled receptors by phosphorylation and endocytosis”, pgs 59-69 Neuropsychopharmacology: the fifth Generation of Progress, incorporated in its entirety herein by reference. Normally GnRH is released from the hypothalamus in a pulsatile fashion and causes a corresponding pulsatile release of LH and FSH. When exogenous GnRH or GnRH agonists are given constantly and in higher than physiologic levels there is an initial significant increase in serum LH, FSH, and sex steroid concentrations. Within approximately two weeks this is followed by a decrease in LH and FSH to undetectable levels resulting in a corresponding decrease in sex steroid production.

As used herein a “therapeutically effective amount” refers to administering a composition in a high enough dosage and for a long enough period of time for the treated individual to experience a decrease in the desire for eating. It is believed that this corresponds to the down regulation of the NPY-1 or NPY-5 receptor (or both) to begin. The time period can also include administering for a length of time after the effect begins and that could include an indefinite time of administration so long as the disease or condition exists. It also assumes continued administration of the composition alone or one or more other therapeutically effective amounts of other compositions that affect appetite so that the receptor continues to remain down regulated during treatment. Should the process be interrupted, the receptors would up regulate to their previous untreated state and the process would need to be started from the beginning. For example, down regulation in most GPCRs of this type can take as long as a couple of weeks before sufficient down regulation occurs that there is consistently a much lower activity. While not wanting to be held to one particular theory, it is believed that any down regulation of the receptors could be potentially employed as a treatment of the present invention. In the end the effect of down regulation seems to concur with the results of the present invention. However, the fact that a decrease in appetite occurs after time with these compositions is the basis of the present invention herein regardless of the theory of how the present invention works.

Continued down regulation of the NPY-1 or NPY-5 receptor refers to the ability of an administered therapy to cause a loss of function of the targeted receptor(s) after an initial period of increased function. In other words, it is felt that the treatment initially causes an activation of the receptors and then, after a period of time with constant treatment, causes the down regulation of the receptors and thus the decrease in appetite effect. The down regulation will be such that effectively a substantial portion, if not all receptors, would be no longer able to signal or be instructed not to signal. By continuing treatment with such compounds, the receptors or their physiological function would not be allowed to return to the normal state or production and signal in the presence of an agonist or other method of stimulating the receptors or mediate their normal physiological effect. It is clear that an important concept in the present invention is the continued treatment by the selected method or composition since discontinuing the treatment could allow the receptors to return to their pretreatment state resulting in a return of excessive appetite, food intake, etc., a normal state and cause appetite enhancement upon retreatment. It has been discovered, as described above, that during this mostly complete down regulation state, that such treatment has an affect similar to treatment with an antagonist. In some embodiments, this effect is without any of the problems associated with competitive antagonist treatments. (One of the problems with antagonists is the loss of effect over time.) In response to the down regulation, the hypothalamus can increase its production and release of peptide in question causing further down regulation. However, when competitive antagonists are used, the increase in hypothalamic peptide production competes for receptor binding and necessitates ever increasing doses of the antagonist.

By “treatment” is meant herein, the administration of a compound which can cause a mammal in need of treatment to have a decrease in appetite after sufficient initial treatment. Therefore, in one embodiment, treatment with NPY, or a composition which causes the over production of NPY or decreased degradation of NPY is administered. In another embodiment, the composition which is administered is a NPY analogue, a NPY-1 agonist or a NPY-5 agonist. In another embodiment, the compositions above are administered for a sufficient time to achieve down regulation or a decrease in appetite. In yet another embodiment, a composition is administered which increases the tendency for the receptor to down regulate.

In use of the present invention, the selected composition would be administered to the patient mammal, such as a human, and the patient in need of treatment and monitored during the phase when appetite would be increased. Thereafter, as the receptors begin to down regulate, the patient would begin to lose the sensation of needing to eat and at the point where the maximum down regulation occurs there would be a significant loss of appetite. One skilled in the art could titrate the composition and determine without undue experimentation the exact dosages as well as the time to achieve down regulation based on size, weight, which mammal, sex, the particular composition and the like, in view of the present disclosure.

“Antagonist(s)” include all agents that interfere with wild-type and/or modified NPY1 or NPY-5 receptor binding to an agonist, and the like, including agents that affect the wild-type and/or modified receptor as well as agents that affect other proteins involved in wild-type and/or modified signaling, and the like.

“Agonists” include all agents that stimulate, cause or bind to the receptors, increased activity of the receptor including signaling, improve signaling, ligand binding and the like.

The phrase “pharmaceutically acceptable” refers to molecular entities and compositions that are physiologically tolerable and do not typically produce an allergic or similar untoward reaction, such as gastric upset, dizziness and the like, when administered to a human.

The term “pharmaceutically acceptable carrier,” as used herein means a pharmaceutically-acceptable material, composition or vehicle, such as a liquid or solid filler, diluent, excipient, solvent or encapsulating material, involved in carrying or transporting a chemical agent.

### Pharmaceutical Compositions

The preparation of compositions which can contain drugs, prodrugs, polypeptides, analogs or active fragments as active ingredients is well understood in the art. The active therapeutic ingredient is often mixed with excipients which are pharmaceutically acceptable and compatible with the active ingredient. Suitable excipients are, for example, water, saline, dextrose, glycerol, ethanol, or the like and combinations thereof. In addition, if desired, the composition can contain minor amounts of auxiliary substances such as wetting or emulsifying agents, pH buffering agents which enhance the effectiveness of the active ingredient.

A composition of the present invention can be formulated into the therapeutic composition as neutralized pharmaceutically acceptable salt forms. Pharmaceutically acceptable salts include the acid addition salts (formed with the free amino groups of the polypeptide or antibody) and which are formed with inorganic acids such as, for example, hydrochloric or phosphoric acids, or such organic acids as acetic, oxalic, tartaric, mandelic, and the like. Salts formed from the free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

When the therapeutic compositions are administered intravenously, intramuscularly, subcutaneously, as by injection, they can be of a unit dose, for example. The term “unit dose” when used in reference to a therapeutic composition of the present invention refers to physically discrete units suitable as unitary dosage for humans, each unit containing a predetermined quantity of active material calculated to produce the desired therapeutic effect in association with the required diluent (i.e., carrier, or vehicle).

The data obtained from cell culture assays and animal studies can be used in formulating a range of dosage for use in humans. The dosage of such compositions lies preferably within a range of circulating concentrations that include the ED50 with little or no toxicity. The dosage may vary within this range depending upon the dosage form employed and the route of administration utilized. For any composition used in the method of the invention, the therapeutically effective dose can be estimated initially from cell culture assays. A dose may be formulated in animal models to achieve a circulating plasma concentration range which includes the 1050 (i.e., the concentration of the test composition which achieves a half-maximal inhibition of symptoms) as determined in cell culture. Such information can be used to more accurately to determine useful doses in humans. Levels in plasma may be measured, for example, by high performance liquid chromatography.

The compositions are administered in a manner compatible with the dosage formulation and in a therapeutically effective amount. The quantity to be administered depends on the subject to be treated, capacity of the subject's system to utilize the active ingredient, and degree of effect desired. Precise amounts of active ingredient required to be administered depend on the judgment of the practitioner and are peculiar to each individual. However, suitable dosages may range from about 0.001 to 30, preferably about 0.01 to about 25, and more preferably about 0.1 to 20 milligrams of active ingredient per kilogram body weight of individual per day and depend on the route of administration. Suitable regimes for initial administration and booster shots are also variable, but are typified by an initial administration followed by repeated doses at one or more hour intervals by a subsequent injection or other administration. Alternatively, continuous intravenous infusion sufficient to maintain concentrations of ten nanomolar to ten micromolar in the blood are contemplated. In yet another embodiment of the present invention the compositions are administered in a depot type formulation. These type formulations, for example using PEG or other compositions for suspending or dispersing a desired composition, are well known. A depot composition can be formulated and placed under the skin or other location. The formulation then releases the desired composition or formulation over a period of time thus creating a form of time release composition which can be used to give a constant dose and help prevent an up regulation due to missed dosages or inadequate dosages.

The skilled artisan will appreciate that certain factors may influence the dosage required to effectively treat a subject, including but not limited to, the severity of the eating related condition or disease, previous treatments, the general health and/or age of the subject, and other diseases present. Moreover, treatment of a subject with a therapeutically effective amount of the composition(s) includes a series of treatments. In a preferred example, a subject is treated with the composition in the range of between about 0.1 to 20 mg/kg body weight, for one time per week to several times per day for between about 1 to 10 weeks. It will preferably be administered as long as necessary to treat the given condition even if that time period is continuously forever. It will also be appreciated that the effective dosage of the composition used for treatment may increase or decrease over the course of a particular treatment. Changes in dosage may result and become apparent from the results of diagnostic assays as described herein. In one embodiment the dosage is administered in a daily continuous type dosage form. The therapeutic compositions may further include an effective amount of the NPY, receptor agonist, and one or more other active ingredients such as other known anti-obesity medications.

The term “prodrug” indicates a therapeutic agent that is prepared in an inactive form that is converted to an active form (i.e., drug) within the body or cells, thereof, by the action of endogenous enzymes or other chemicals and/or conditions. In particular, prodrug versions of the compositions of the invention can be prepared as SATE [(S-acetyl-2-thioethyl)phosphate] derivatives according to the methods disclosed, for example, in WO 93/24510 and in WO 94/26764.

The term “pharmaceutically acceptable salts” refers to physiologically and pharmaceutically acceptable salts of the compounds of the invention: i.e., salts that retain the desired biological activity of the parent compound and do not impart undesired toxicological effects thereto.

Pharmaceutically acceptable base addition salts for use compositions of the invention are formed with metals or amines, such as alkali and alkaline earth metals or organic amines. Examples of metals used as cations are sodium, potassium, magnesium, calcium, and the like. Examples of suitable amines are N,N′-dibenzylethylenediamine, chloroprocaine, choline, diethanolamine, dicyclohexylamine, ethylenediamine, N-methylglucamine, and procaine (see, e.g., Berge et al., “Pharmaceutical Salts,” J. Pharma. Sci., 1977, 66: 119). The base addition salts of acidic compounds are prepared by contacting the free acid form with a sufficient amount of the desired base to produce the salt in the conventional manner. The free acid form may be regenerated by contacting the salt form with an acid and isolating the free acid in the conventional manner. The free acid forms differ from their respective salt forms somewhat in certain physical properties, such as solubility in polar solvents, but otherwise the salts are equivalent to their respective free acid for purposes of the present invention. As used herein, a “pharmaceutical addition salt” includes a pharmaceutically acceptable salt of an acid form of one of the components of the compositions of the invention. These include organic or inorganic acid salts of the amines. Preferred acid salts are the hydrochlorides, acetates, salicylates, nitrates and phosphates. Other suitable pharmaceutically acceptable salts are known in the art and include basic salts of a variety of inorganic and organic acids, such as, for example, with inorganic acids (e.g., hydrochloric acid, hydrobromic acid, sulfuric acid or phosphoric acid); with organic carboxylic, sulfonic, sulfo- or phospho-acids or N-substituted sulfamic acids, for example acetic acid, propionic acid, glycolic acid, succinic acid, maleic acid, hydroxymaleic acid, methylmaleic acid, fumaric acid, malic acid, tartaric acid, lactic acid, oxalic acid, gluconic acid, glucaric acid, glucuronic acid, citric acid, benzoic acid, cinnamic acid, mandelic acid, salicylic acid, 4-aminosalicylic acid, 2-phenoxybenzoic acid, 2-acetoxybenzoic acid, embonic acid, nicotinic acid or isonicotinic acid; and with amino acids, such as the 20 alpha-amino acids involved in the synthesis of proteins in nature, for example glutamic acid or aspartic acid, and also with phenylacetic acid, methanesulfonic acid, ethanesulfonic acid, 2-hydroxyethanesulfonic acid, ethane-1,2-disulfonic acid, benzenesulfonic acid, 4-methylbenzenesulfonic acid, naphthalene-2-sulfonic acid, naphthalene-1,5-disulfonic acid, 2- or 3-phosphoglycerate, glucose-6-phosphate, N-cyclohexylsulfamic acid (with the formation of cyclamates), or with other acid organic compounds, such as ascorbic acid.

Pharmaceutically acceptable salts of compounds may also be prepared with a pharmaceutically acceptable cation. Suitable pharmaceutically acceptable cations are well known in the art and include alkaline, alkaline earth, ammonium and quaternary ammonium cations. Carbonates or hydrogen carbonates are also possible.

For topical application, the compositions may be combined with a carrier so that an effective dosage is delivered, based on the desired activity. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

For oral administration, the pharmaceutical acceptable compositions may take the form of, for example, tablets or capsules prepared by conventional means with pharmaceutically acceptable excipients, such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer, salts, flavoring, coloring and sweetening agents as appropriate. Preparations for oral administration may be suitably formulated to give controlled release of the active composition.

The compositions may be formulated for parenteral administration by injection, e.g., by bolus injection or continuous infusion. Formulations for injection may be presented in unit dosage form, e.g., in ampules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

For administration by inhalation, the compositions for use according to the present invention are conveniently delivered in the form of an aerosol spray, presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Capsules and cartridges of, e.g., gelatin for use in an inhaler or insufflator may be formulated containing a powder mix of the composition and a suitable powder base such as lactose or starch.

The compositions may also be formulated as a depot preparation. Such long acting formulations may be administered by implantation (for example subcutaneously or intramuscularly) or by intramuscular injection. Thus, for example, the compositions may be formulated with suitable polymeric or hydrophobic materials (for example, as an emulsion in an acceptable oil) or ion exchange resins, or as sparingly soluble derivatives, for example, as a sparingly soluble salt.

Even further the composition may be formulated into an osmotic pump using those means known in the art.

The compositions may, if desired, be presented in a pack or dispenser device that may contain one or more unit dosage forms containing the active ingredient. The pack may, for example, comprise metal or plastic foil, such as a blister pack. The pack or dispenser device may be accompanied by instructions for administration.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general, the formulations are prepared by uniformly and intimately bringing into association the active ingredients with liquid carriers or finely divided solid carriers or both, and then, if necessary, shaping the product.

In one embodiment of the present invention, the pharmaceutical compositions may be formulated and used as foams. Pharmaceutical foams include formulations such as, but not limited to, emulsions, microemulsions, creams, jellies and liposomes. While basically similar in nature, these formulations vary in the components and the consistency of the final product. The preparation of such compositions and formulations is generally known to those skilled in the pharmaceutical and formulation arts and may be applied to the formulation of the compositions of the present invention.

The compositions of the present invention may be prepared and formulated as emulsions. Emulsions are typically heterogenous systems of one liquid dispersed in another in the form of droplets usually exceeding 0.1 m in diameter. See, e.g., Idson, in Pharmaceutical Dosage Forms v. 1, p. 199 (Lieberman, Rieger and Banker (Eds.), 1988, Marcel Dekker, Inc., New York); Rosoff, in Pharmaceutical Dosage Forms, v. 1, p. 245; Block in Pharmaceutical Dosage Forms, v. 2, p. 335; Higuchi et al., in Remington's Pharmaceutical Sciences 301 (Mack Publishing Co., Easton, Pa., 1985). Emulsions are often biphasic systems comprising of two immiscible liquid phases intimately mixed and dispersed with each other. In general, emulsions may be either water-in-oil (w/o) or of the oil-in-water (o/w) variety. When an aqueous phase is finely divided into and dispersed as minute droplets into a bulk oily phase, the resulting composition is called a water-in-oil (w/o) emulsion. Alternatively, when an oily phase is finely divided into and dispersed as minute droplets into a bulk aqueous phase the resulting composition is called an oil-in-water (o/w) emulsion. Emulsions may contain additional components in addition to the dispersed phases and the active drug which may be present as a solution in either the aqueous phase, oily phase or itself as a separate phase. Pharmaceutical excipients such as emulsifiers, stabilizers, dyes, and anti-oxidants may also be present in emulsions as needed. Pharmaceutical emulsions may also be multiple emulsions that are comprised of more than two phases such as, for example, in the case of oil-in-water-in-oil (o/w/o) and water-in-oil-in-water (w/o/w) emulsions. Such complex formulations often provide certain advantages that simple binary emulsions do not. Multiple emulsions in which individual oil droplets of an o/w emulsion enclose small water droplets constitute a w/o/w emulsion. Likewise a system of oil droplets enclosed in globules of water stabilized in an oily continuous provides an o/w/o emulsion.

Emulsions are characterized by little or no thermodynamic stability. Often, the dispersed or discontinuous phase of the emulsion is well dispersed into the external or continuous phase and maintained in this form through the means of emulsifiers or the viscosity of the formulation. Either of the phases of the emulsion may be a semisolid or a solid, as is the case of emulsion-style ointment bases and creams. Other means of stabilizing emulsions entail the use of emulsifiers that may be incorporated into either phase of the emulsion. Emulsifiers may broadly be classified into four categories: synthetic surfactants, naturally occurring emulsifiers, absorption bases, and finely dispersed solids (Idson, in Pharmaceutical Dosage Forms v. 1, p. 199 [Lieberman, Rieger and Banker (Eds.), 1988, Marcel Dekker, Inc., New York]).

Synthetic surfactants, also known as surface active agents, have found wide applicability in the formulation of emulsions and have been reviewed in the literature (Rieger, in Pharmaceutical Dosage Forms, v. 1, p. 285; Idson, in Pharmaceutical Dosage Forms, v. 1, p. 199). Surfactants are typically amphiphilic and comprise a hydrophilic and a hydrophobic portion. The ratio of the hydrophilic to the hydrophobic nature of the surfactant has been termed the hydrophile/lipophile balance (HLB) and is a valuable tool in categorizing and selecting surfactants in the preparation of formulations. Surfactants may be classified into different classes based on the nature of the hydrophilic group: nonionic, anionic, cationic and amphoteric (Rieger, in Pharmaceutical Dosage Forms).

Naturally occurring emulsifiers used in emulsion formulations include lanolin, beeswax, phosphatides, lecithin and acacia. Absorption bases possess hydrophilic properties such that they can soak up water to form w/o emulsions yet retain their semisolid consistencies, such as anhydrous lanolin and hydrophilic petrolatum. Finely divided solids have also been used as good emulsifiers, especially in combination with surfactants and in viscous preparations. These include polar inorganic solids, such as heavy metal hydroxides, non-swelling clays (e.g., bentonite, attapulgite, hectorite, kaolin, montmorillonite, colloidal aluminum silicate and colloidal magnesium aluminum silicate), pigments and nonpolar solids (e.g., carbon or glyceryl tristearate).

A large variety of non-emulsifying materials can also be included in emulsion formulations and contribute to the properties of emulsions. These include fats, oils, waxes, fatty acids, fatty alcohols, fatty esters, humectants, hydrophilic colloids, preservatives and antioxidants (Block, in Pharmaceutical Dosage Forms, v. 1 p. 385 [Lieberman, Rieger and Banker (Eds.), 1988, Marcel Dekker, Inc., New York)].

Hydrophilic colloids or hydrocolloids include naturally occurring gums and synthetic polymers, such as polysaccharides (e.g., acacia, agar, alginic acid, carrageenan, guar gum, karaya gum, and tragacanth), cellulose derivatives (e.g., carboxymethylcellulose and carboxypropylcellulose), and synthetic polymers (e.g., carbomers, cellulose ethers, and carboxyvinyl polymers). These disperse or swell in water to form colloidal solutions that stabilize emulsions by forming strong interfacial films around the dispersed-phase droplets and by increasing the viscosity of the external phase.

Since emulsions often contain a number of ingredients such as carbohydrates, proteins, sterols and phosphatides that may readily support the growth of microbes, these formulations often incorporate preservatives. Commonly used preservatives included in emulsion formulations include methyl paraben, propyl paraben, quaternary ammonium salts, benzalkonium chloride, esters of p-hydroxybenzoic acid, and boric acid. Antioxidants are also commonly added to emulsion formulations to prevent deterioration of the formulation. Antioxidants used may be free radical scavengers (e.g., tocopherols, alkyl gallates, butylated hydroxyanisole, butylated hydroxytoluene) or reducing agents (e.g., ascorbic acid and sodium metabisulfite), and antioxidant synergists (e.g., citric acid, tartaric acid, and lecithin).

The application of emulsion formulations via dermatological, oral and parenteral routes and the methods for their manufacture have been reviewed in the literature (Idson, in Pharmaceutical Dosage Forms, v. 1, p. 199). Emulsion formulations for oral delivery have been very widely used because of reasons of ease of formulation, efficacy from an absorption and bioavailability standpoint. [Rosoff, in Pharmaceutical Dosage Forms, v. 1, p. 245 (Lieberman, Rieger and Banker (Eds.), 1988, Marcel Dekker, Inc., New York); Idson, in Pharmaceutical Dosage Forms]. Mineral-oil base laxatives, oil-soluble vitamins and high fat nutritive preparations are among the materials that have commonly been administered orally as o/w emulsions.

In one embodiment of the present invention, the compositions are formulated as microemulsions. A microemulsion may be defined as a system of water, oil and amphiphile which is a single optically isotropic and thermodynamically stable liquid solution (Rosoff, in Pharmaceutical Dosage Forms, v. 1, p. 245). Typically microemulsions are systems that are prepared by first dispersing an oil in an aqueous surfactant solution and then adding a sufficient amount of a fourth component, generally an intermediate chain-length alcohol to form a transparent system. Therefore, microemulsions have also been described as thermodynamically stable, isotropically clear dispersions of two immiscible liquids that are stabilized by interfacial films of surface-active molecules (Leung and Shah, in Controlled Release of Drugs: Polymers and Aggregate Systems, 185 215 (Rosoff, M., Ed., 1989, VCH Publishers, New York). Microemulsions are commonly prepared via a combination of three to five components that include oil, water, surfactant, cosurfactant and electrolyte. Whether the microemulsion is of the water-in-oil (w/o) or an oil-in-water (o/w) type is dependent on the properties of the oil and surfactant used and on the structure and geometric packing of the polar heads and hydrocarbon tails of the surfactant molecules (Schott, in Remington's Pharmaceutical Sciences, 271 (Mack Publishing Co., Easton, Pa., 1985).

Surfactants used in the preparation of microemulsions include, but are not limited to, ionic surfactants, non-ionic surfactants, Brij 96, polyoxyethylene oleyl ethers, polyglycerol fatty acid esters, tetraglycerol monolaurate (ML310), tetraglycerol monooleate (MO310), hexaglycerol monooleate (PO310), hexaglycerol pentaoleate (PO500), decaglycerol monocaprate (MCA750), decaglycerol monooleate (MO750), decaglycerol sequioleate (SO750), decaglycerol decaoleate (DAO750), alone or in combination with co-surfactants. The co-surfactant, usually a short-chain alcohol such as ethanol, 1-propanol, and 1-butanol, serves to increase the interfacial fluidity by penetrating into the surfactant film and consequently creating a disordered film because of the void space generated among surfactant molecules.

Microemulsions may, however, be prepared without the use of co-surfactants and alcohol-free self-emulsifying microemulsion systems are known in the art. The aqueous phase may typically be, but is not limited to, water, an aqueous solution of the drug, glycerol, PEG300, PEG400, polyglycerols, propylene glycols, and derivatives of ethylene glycol. The oil phase may include, but is not limited to, materials such as Captex 300, Captex 355, Capmul MOM, fatty acid esters, medium chain (C8 C12) mono-, di-, and tri-glycerides, polyoxyethylated glyceryl fatty acid esters, fatty alcohols, polyglycolized glycerides, saturated polyglycolized C8 C10 glycerides, vegetable oils and silicone oil.

Microemulsions are particularly of interest from the standpoint of drug solubilization and the enhanced absorption of drugs. Lipid based microemulsions (both o/w and w/o) have been proposed to enhance the oral bioavailability of drugs, including peptides (Constantinides et al., Pharm. Res., 1994, 11:1385 90; Ritschel, Meth. Find. Exp. Clin. Pharmacol., 1993, 13: 205). Microemulsions afford advantages of improved drug solubilization, protection of drug from enzymatic hydrolysis, possible enhancement of drug absorption due to surfactant-induced alterations in membrane fluidity and permeability, ease of preparation, ease of oral administration over solid dosage forms, improved clinical potency, and decreased toxicity (Constantinides et al., 1994; Ho et al., J. Pharm. Sci., 1996, 85: 138 143). Often microemulsions may form spontaneously when their components are brought together at ambient temperature. This may be particularly advantageous when formulating thermolabile drugs, peptides or oligonucleotides. Microemulsions have also been effective in the transdermal delivery of active components in both cosmetic and pharmaceutical applications. It is expected that the microemulsion compositions and formulations of the present invention will facilitate the increased systemic absorption of oligonucleotides and nucleic acids and other active agents from the gastrointestinal tract, as well as improve the local cellular uptake of oligonucleotides and nucleic acids and other active agents within the gastrointestinal tract, vagina, buccal cavity and other areas of administration.

Microemulsions of the present invention may also contain additional components and additives such as sorbitan monostearate (Grill 3), Labrasol, and penetration enhancers to improve the properties of the formulation and to enhance the absorption of the oligonucleotides and nucleic acids of the present invention. Penetration enhancers used in the microemulsions of the present invention may be classified as belonging to one of five broad categories—surfactants, fatty acids, bile salts, chelating agents, and non-chelating non-surfactants (Lee et al., Crit. Rev. Therap. Drug Carrier Systems, 1991, p. 92). Each of these classes have been discussed above.

There are many organized surfactant structures besides microemulsions that have been studied and used for the formulation of drugs. These include monolayers, micelles, bilayers and vesicles. Vesicles, such as liposomes, are useful because of their specificity and the duration of action. As used in the present invention, the term “liposome” means a vesicle composed of amphiphilic lipids arranged in a spherical bilayer or bilayers.

Liposomes are unilamellar or multilamellar vesicles which have a membrane formed from a lipophilic material and an aqueous interior. The aqueous portion contains the composition to be delivered. Cationic liposomes possess the advantage of being able to fuse to the cell wall. Non-cationic liposomes, although not able to fuse as efficiently with the cell wall, are taken up by macrophages in vivo. Selection of the appropriate liposome depending on the agent to be encapsulated would be evident given what is known in the art.

In order to cross intact mammalian skin, lipid vesicles must pass through a series of fine pores, each with a diameter less than 50 nm, under the influence of a suitable transdermal gradient. Therefore, it is desirable to use a liposome which is highly deformable and able to pass through such fine pores.

Further advantages of liposomes include: (a) liposomes obtained from natural phospholipids are biocompatible and biodegradable; (b) liposomes can incorporate a wide range of water and lipid soluble drugs; (c) liposomes can protect encapsulated drugs in their internal compartments from metabolism and degradation (Rosoff, in Pharmaceutical Dosage Forms). Important considerations in the preparation of liposome formulations are the lipid surface charge, vesicle size and the aqueous volume of the liposomes.

Liposomes are useful for the transfer and delivery of active ingredients to the site of action. Because the liposomal membrane is structurally similar to biological membranes, when liposomes are applied to a tissue, the liposomes start to merge with the cellular membranes. As the merging of the liposome and cell progresses, the liposomal contents are emptied into the cell where the active agent may act.

Another embodiment also contemplates the use of liposomes for topical administration. Such advantages include reduced side-effects related to high systemic absorption of the administered drug, increased accumulation of the administered drug at the desired target, and the ability to administer a wide variety of drugs, both hydrophilic and hydrophobic, into the skin. Several reports have detailed the ability of liposomes to deliver agents including high-molecular weight DNA into the skin. Compounds including analgesics, antibodies, hormones and high-molecular weight DNAs have been administered to the skin. The majority of applications resulted in the targeting of the upper epidermis.

Liposomes fall into two broad classes. Cationic liposomes are positively charged liposomes which interact with the negatively charged DNA molecules to form a stable complex. The positively charged DNA/liposome complex binds to the negatively charged cell surface and is internalized in an endosome. Due to the acidic pH within the endosome, the liposomes are ruptured, releasing their contents into the cell cytoplasm (Wang et al., Biochem. Biophys. Res. Comm., 1987, 147:980 985).

Liposomes which are pH-sensitive or negatively-charged, entrap DNA rather than complex with it. Since both the DNA and the lipid are similarly charged, repulsion rather than complex formation occurs. Nevertheless, some DNA is entrapped within the aqueous interior of these liposomes. pH-sensitive liposomes have been used to deliver DNA encoding the thymidine kinase gene to cell monolayers in culture. Expression of the exogenous gene was detected in the target cells (Zhou et al., J. Controlled Release, 1992, 19: 269 74).

Another contemplated liposomal composition includes phospholipids other than naturally-derived phosphatidylcholine. Neutral liposome compositions, for example, can be formed from dimyristoyl phosphatidylcholine (DMPC) or dipalmitoyl phosphatidylcholine (DPPC). Anionic liposome compositions generally are formed from dimyristoyl phosphatidylglycerol, while anionic fusogenic liposomes are formed primarily from dioleoyl phosphatidylethanolamine (DOPE). Another type of liposomal composition is formed from phosphatidylcholine (PC) such as, for example, soybean PC, and egg PC. Another type is formed from mixtures of phospholipid and/or phosphatidylcholine and/or cholesterol.

“Sterically stabilized” liposomes, which refers to liposomes comprising one or more specialized lipids that, when incorporated into liposomes, result in enhanced circulation lifetimes relative to liposomes lacking such specialized lipids are also contemplated. Examples of sterically stabilized liposomes are those in which part of the vesicle-forming lipid portion of the liposome (A) comprises one or more glycolipids, such as monosialoganglioside GM1, or (B) is derivatized with one or more hydrophilic polymers, such as a polyethylene glycol (PEG) moiety. While not wishing to be bound by any particular theory, it is thought in the art that, at least for sterically stabilized liposomes containing gangliosides, sphingomyelin, or PEG-derivatized lipids, the enhanced circulation half-life of these sterically stabilized liposomes derives from a reduced uptake into cells of the reticuloendothelial system (RES) (Allen et al., FEBS Lett., 1987, 223: 42; Wu et al., Can. Res., 1993, 53: 3765).

Many liposomes comprising lipids derivatized with one or more hydrophilic polymers, and methods of preparation thereof, are known in the art. See, e.g., Sunamoto et al. (Bull. Chem. Soc. Jpn., 1980, 53: 2778) described liposomes comprising a nonionic detergent, 2C12 15G that contains a PEG moiety. Illium et al. (FEBS Lett., 1984, 167: 79) noted that hydrophilic coating of polystyrene particles with polymeric glycols results in significantly enhanced blood half-lives. Synthetic phospholipids modified by the attachment of carboxylic groups of polyalkylene glycols (e.g., PEG) are described by Sears (U.S. Pat. Nos. 4,426,330 and 4,534,899). Klibanov et al. (FEBS Lett., 1990, 268: 235) described experiments demonstrating that liposomes comprising phosphatidylethanolamine (PE) derivatized with PEG or PEG stearate have significant increases in blood circulation half-lives. Blume et al. (Biochimica et Biophysica Acta, 1990, 1029: 91) extended such observations to other PEG-derivatized phospholipids, e.g., DSPE-PEG, formed from the combination of distearoylphosphatidylethanolamine (DSPE) and PEG. Liposomes having covalently bound PEG moieties on their external surface are described in European Patent No. EP 0 445 131 B1 and WO 90/04384 to Fisher. Liposome compositions containing 1 20 mole percent of PE derivatized with PEG, and methods of use thereof, are described by, e.g., Woodle et al. (U.S. Pat. Nos. 5,013,556 and 5,356,633) and Martin et al. (U.S. Pat. No. 5,213,804 and European Patent No. EP 0 496 813 B1). Liposomes comprising a number of other lipid-polymer conjugates are disclosed in WO 91/05545 and U.S. Pat. No. 5,225,212 (both to Martin et al.) and in WO 94/20073 (Zalipsky et al.). Liposomes comprising PEG-modified ceramide lipids are described in WO 96/10391 (Choi et al.). U.S. Pat. No. 5,540,935 (Miyazaki et al.) and U.S. Pat. No. 5,556,948 (Tagawa et al.) describe PEG-containing liposomes that can be further derivatized with functional moieties on their surfaces.

Methods of encapsulating nucleic acids in liposomes are also known in the art. See, WO 96/40062 to Thierry et al. discloses methods for encapsulating high molecular weight nucleic acids in liposomes. U.S. Pat. No. 5,264,221 to Tagawa et al. discloses protein-bonded liposomes and asserts that the contents of such liposomes may include an antisense RNA. U.S. Pat. No. 5,665,710 to Rahman et al. describes certain methods of encapsulating oligodeoxynucleotides in liposomes.

Surfactants find wide application in formulations such as emulsions (including microemulsions) and liposomes. The most common way of classifying and ranking the properties of the many different types of surfactants, both natural and synthetic, is by the use of the hydrophile/lipophile balance (HLB). The nature of the hydrophilic group (also known as the “head”) provides the most useful means for categorizing the different surfactants used in formulations (Rieger, in Pharmaceutical Dosage Forms, p. 285 (Marcel Dekker, Inc., New York, N.Y., 1988, p. 285)).

If the surfactant molecule is not ionized, it is classified as a nonionic surfactant. Nonionic surfactants find wide applications in pharmaceutical and cosmetic products and are usable over a wide range of pH values. In general their HLB values range from 2 to about 18 depending on their structure. Nonionic surfactants include nonionic esters such as ethylene glycol esters, propylene glycol esters, glyceryl esters, polyglyceryl esters, sorbitan esters, sucrose esters, and ethoxylated esters. Nonionic alkanolamides and ethers such as fatty alcohol ethoxylates, propoxylated alcohols, and ethoxylated/propoxylated block polymers are also included in this class. The polyoxyethylene surfactants are the most popular members of the nonionic surfactant class.

If the surfactant molecule carries a negative charge when it is dissolved or dispersed in water, the surfactant is classified as anionic. Anionic surfactants include carboxylates such as soaps, acyl lactylates, acyl amides of amino acids, esters of sulfuric acid such as alkyl sulfates and ethoxylated alkyl sulfates, sulfonates such as alkyl benzene sulfonates, acyl isethionates, acyl taurates and sulfosuccinates, and phosphates. The most important members of the anionic surfactant class are the alkyl sulfates and the soaps.

If the surfactant molecule carries a positive charge when it is dissolved or dispersed in water, the surfactant is classified as cationic. Cationic surfactants include quaternary ammonium salts and ethoxylated amines. The quaternary ammonium salts are the most used members of this class.

If the surfactant molecule has the ability to carry either a positive or negative charge, the surfactant is classified as amphoteric. Amphoteric surfactants include acrylic acid derivatives, substituted alkylamides, N-alkylbetaines and phosphatides.

The use of surfactants in drug products, formulations and in emulsions has been reviewed [Rieger, in Pharmaceutical Dosage Forms, 285 (Marcel Dekker, Inc., New York, N.Y., 1988)].

In one embodiment, the present invention employs various penetration enhancers to effect the efficient delivery of nucleic acids and other agents, particularly oligonucleotides, to the skin of animals. Most drugs are present in solutions in both ionized and nonionized forms. However, usually only lipid soluble or lipophilic drugs readily cross cell membranes. It has been discovered that even non-lipophilic drugs may cross cell membranes if the membrane to be crossed is treated with a penetration enhancer. In addition to aiding the diffusion of non-lipophilic drugs across cell membranes, penetration enhancers also enhance the permeability of lipophilic drugs.

Penetration enhancers may be classified as belonging to one of five broad categories, i.e., surfactants, fatty acids, bile salts, chelating agents, and non-chelating non-surfactants (Lee et al., Critical Reviews in Therapeutic Drug Carrier Systems, 1991, p. 92). Each of the above mentioned classes of penetration enhancers are described below in greater detail.

Another embodiment of the invention contemplates pharmaceutical compositions comprising surfactants. Surfactants (or “surface-active agents”) are chemical entities which, when dissolved in an aqueous solution, reduce the surface tension of the solution or the interfacial tension between the aqueous solution and another liquid, with the result that absorption of oligonucleotides through the mucosa is enhanced. In addition to bile salts and fatty acids, these penetration enhancers include, for example, sodium lauryl sulfate, polyoxyethylene-9-lauryl ether and polyoxyethylene-20-cetyl ether) (Lee et al., Crit. Rev. Therap. Drug Carrier Systems, 1991, 92); and perfluorochemical emulsions, such as FC-43 (Takahashi et al., J. Pharm. Pharmacol., 1988, 40: 252).

Another embodiment contemplates the use of various fatty acids and their derivatives to act as penetration enhancers include, for example, oleic acid, lauric acid, capric acid (n-decanoic acid), myristic acid, palmitic acid, stearic acid, linoleic acid, linolenic acid, dicaprate, tricaprate, monoolein (1-monooleoyl-rac-glycerol), dilaurin, caprylic acid, arachidonic acid, glycerol 1-monocaprate, 1-dodecylazacycloheptan-2-one, acylcarnitines, acylcholines, C1 10 alkyl esters thereof (e.g., methyl, isopropyl and t-butyl), and mono- and di-glycerides thereof (i.e., oleate, laurate, caprate, myristate, palmitate, stearate, linoleate, and the like) (Lee et al., 1991; Muranishi, Crit. Rev. Therap. Drug Carrier Systems, 1990, 7: 1 33; El Hariri et al., J. Pharm. Pharmacol., 1992, 44: 651 4).

The compositions comprising the active agents of the invention may further comprise bile salts. The physiological role of bile includes the facilitation of dispersion and absorption of lipids and fat-soluble vitamins (Brunton, Chapter 38 in: Goodman & Gilman's The Pharmacological Basis of Therapeutics, 9th Ed., Hardman et al. Eds., McGraw-Hill, N.Y., 1996, pp. 934-935). Various natural bile salts, and their synthetic derivatives, act as penetration enhancers. Thus, the term “bile salts” includes any of the naturally occurring components of bile as well as any of their synthetic derivatives. The bile salts of the invention include, for example, cholic acid (or its pharmaceutically acceptable sodium salt, sodium cholate), dehydrocholic acid (sodium dehydrocholate), deoxycholic acid (sodium deoxycholate), glucholic acid (sodium glucholate), glycholic acid (sodium glycocholate), glycodeoxycholic acid (sodium glycodeoxycholate), taurocholic acid (sodium taurocholate), taurodeoxycholic acid (sodium taurodeoxycholate), chenodeoxycholic acid (sodium chenodeoxycholate), ursodeoxycholic acid (UDCA), sodium tauro-24, 25-dihydro-fusidate (STDHF), sodium glycodihydrofusidate and polyoxyethylene-9-lauryl ether (POE) (Lee et al., 1991; Swinyard, Chapter 39 In: Remington's Pharmaceutical Sciences, 18th Ed., Gennaro, ed., Mack Publishing Co., Easton, Pa., 1990, pages 782 783; Muranishi, 1990; Yamamoto et al., J. Pharm. Exp. Ther., 1992, 263: 25; Yamashita et al., J. Pharm. Sci., 1990, 79: 579 83).

The invention further contemplates compositions comprising chelating agents. Chelating agents can be defined as compounds that remove metallic ions from solution by forming complexes therewith, with the result that absorption of oligonucleotides through the mucosa is enhanced. With regards to their use as penetration enhancers for use when the active agent is an antisense agent, chelating agents have the added advantage of also serving as DNase inhibitors, as most characterized DNA nucleases require a divalent metal ion for catalysis and are thus inhibited by chelating agents (Jarrett, J. Chromatogr., 1993, 618: 315 39). Chelating agents of the invention include, but are not limited to, disodium ethylenediaminetetraacetate (EDTA), citric acid, salicylates (e.g., sodium salicylate, 5-methoxysalicylate and homovanilate), N-acyl derivatives of collagen, laureth-9 and N-amino acyl derivatives of beta-diketones (enamines) (Lee et al., 1991; Muranishi, 1990; Buur et al., J. Control Rel., 1990, 14:43 51).

The invention also contemplates pharmaceutical compositions comprising active agents and non-chelating non-surfactants. Non-chelating non-surfactant penetration enhancing compounds can be defined as compounds that demonstrate insignificant activity as chelating agents or as surfactants, but that nonetheless enhance absorption of oligonucleotides through the alimentary mucosa (Muranishi, 1990). This class of penetration enhancers include, for example, unsaturated cyclic ureas, 1-alkyl- and 1-alkenylazacyclo-alkanone derivatives (Lee et al., 1991); and non-steroidal anti-inflammatory agents such as diclofenac sodium, indomethacin and phenylbutazone (Yamashita et al., J. Pharm. Pharmacol., 1987, 39: 621 6).

Other agents may be utilized to enhance the penetration of the administered compositions, including glycols such as ethylene glycol and propylene glycol, pyrrols such as 2-pyrrol, azones, and terpenes (e.g., limonene and menthone).

Certain compositions of the present invention also incorporate carrier compounds in the formulation. As used herein, “carrier compound” or “carrier” can refer to a nucleic acid, or analog thereof, which is inert (i.e., does not possess biological activity per se) but is recognized as a nucleic acid by in vivo processes that reduce the bioavailability of a nucleic acid having biological activity by, for example, degrading the biologically active nucleic acid or promoting its removal from circulation. The coadministration of a nucleic acid and a carrier compound, typically with an excess of the latter substance, can result in a substantial reduction of the amount of nucleic acid recovered in the liver, kidney or other extracirculatory reservoirs, presumably due to competition between the carrier compound and the nucleic acid for a common receptor. For example, the recovery of a partially phosphorothioate oligonucleotide in hepatic tissue can be reduced when it is coadministered with polyinosinic acid, dextran sulfate, polycytidic acid or 4-acetamido-4′-isothiocyano-stilbene-2,2′-disulfonic acid (Miyao et al., Antisense Res. Dev., 1995, 5: 115 121; Takakura et al., Antisense & Nucl. Acid Drug Dev., 1996, 6: 177 183).

The pharmaceutical compositions disclosed herein may also comprise excipients. In contrast to carrier compounds described above, these excipients include a pharmaceutically acceptable solvent, suspending agent or any other pharmacologically inert vehicle for delivering one or more peptides or other active agents to an animal. The excipient may be liquid or solid and is selected, with the planned manner of administration in mind, so as to provide for the desired bulk, consistency, etc., when combined with a nucleic acid or other active agent and the other components of a given pharmaceutical composition. Typical pharmaceutical carriers include, but are not limited to, binding agents (e.g., pregelatinized maize starch, polyvinylpyrrolidone or hydroxypropyl methylcellulose, etc.); fillers (e.g., lactose and other sugars, microcrystalline cellulose, pectin, gelatin, calcium sulfate, ethyl cellulose, polyacrylates or calcium hydrogen phosphate, etc.); lubricants (e.g., magnesium stearate, talc, silica, colloidal silicon dioxide, stearic acid, metallic stearates, hydrogenated vegetable oils, corn starch, polyethylene glycols, sodium benzoate, sodium acetate, etc.); disintegrants (e.g., starch, sodium starch glycolate, etc.); and wetting agents (e.g., sodium lauryl sulphate, etc.).

Pharmaceutically acceptable organic or inorganic excipients suitable for non-parenteral administration which do not deleteriously react with peptides can also be used to formulate the compositions of the present invention. Suitable pharmaceutically acceptable carriers include, but are not limited to, water, salt solutions, alcohols, polyethylene glycols, gelatin, lactose, amylose, magnesium stearate, talc, silicic acid, viscous paraffin, hydroxymethylcellulose, polyvinylpyrrolidone and the like.

Formulations for topical administration of nucleic acids and other contemplated active agents may include sterile and non-sterile aqueous solutions, non-aqueous solutions in common solvents such as alcohols, or solutions of the nucleic acids in liquid or solid oil bases. The solutions may also contain buffers, diluents and other suitable additives. Pharmaceutically acceptable organic or inorganic excipients suitable for non-parenteral administration which do not deleteriously react with nucleic acids or other contemplated active agents can be used.

The compositions of the present invention may additionally contain other adjunct components conventionally found in pharmaceutical compositions, at their art-established usage levels. Thus, for example, the compositions may contain additional, compatible, pharmaceutically-active materials such as, e.g., antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like, which do not deleteriously interact with the nucleic acid(s) of the formulation.

Aqueous suspensions may contain substances which increase the viscosity of the suspension including, for example, sodium carboxymethylcellulose, sorbitol and/or dextran. The suspension may also contain stabilizers.

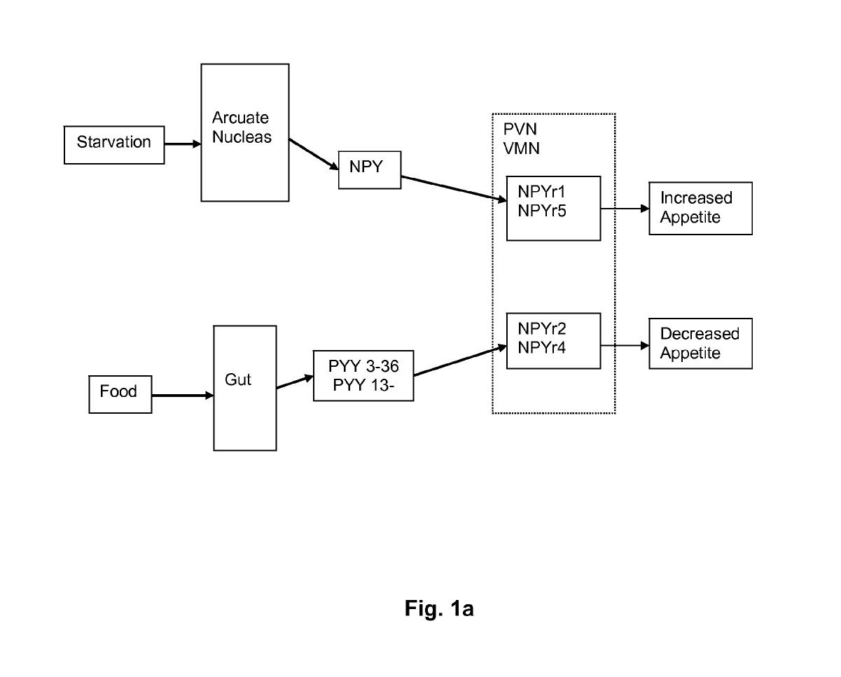

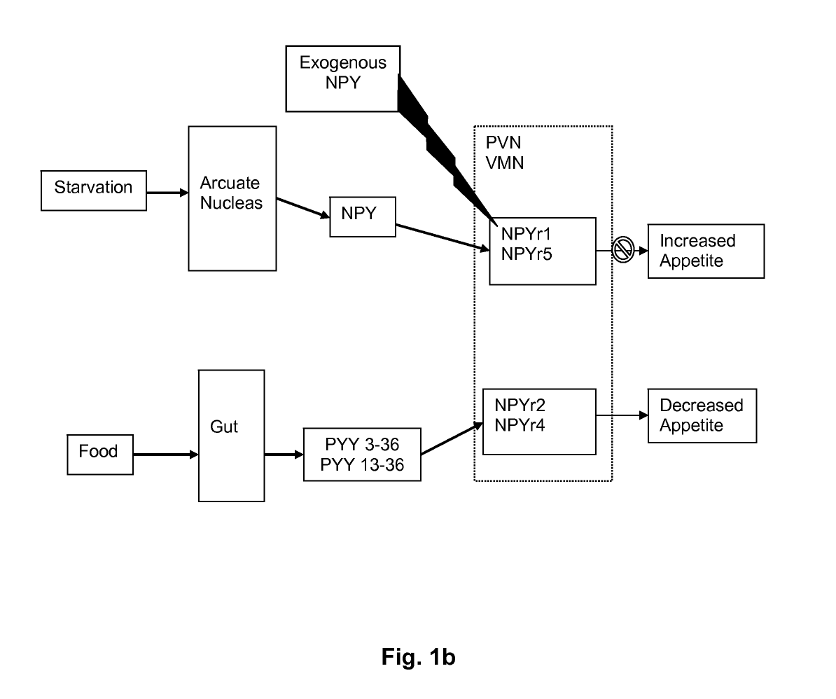

Now referring to the figures, FIG. 1a depicts the known relationship of appetite suppression and stimulation with NPY receptors. Starvation stimulates the arcuate nucleas in the hypothalamus to secrete NPY. NPY then binds to NPY1 and NPY5 receptors in the paraventricular nucleas (PVM) and the ventromedial nucleas (VMN) which stimulates appetite and food intake. Food intake results in loss of starvation stimuli to the arcuate nucleas plus causes the gut to secrete PYY 3-36 and PYY 13-36. These bind to NPY2 and NPY4 receptors in the paraventricular nucleas and ventromedial nucleas which causes and inhibition of appetite.

FIG. 1b depicts a flow diagram of the method of activity of the present invention regulation of the NPY receptors. High dose continuous exogenous NPY causes an initial stimulation of appetite via signaling through NPYr1 and NPYr5 receptors. However, this is followed by down regulation of these receptors resulting in a loss of appetite stimulation. It leaves intact the pathway involving PYY 3-36/PYY 13-36 and the NPYr2/NPYr4 receptors which inhibit appetite.

The above examples and embodiments are intended to teach one skilled in the art the workings of the invention. They are not intended to be limiting in scope. Other choices of down regulation control, compositions formulations and the like will be obvious to one skilled in the art and the claims which follow are to be so interpreted.

What is claimed is:

1. A method of treating an eating related disease or condition in a mammal in need of such treatment comprising administering to the mammal a therapeutically effective amount of a pharmaceutically acceptable composition selected from the group comprising NPY, a NPY stimulant, a NPY degradation inhibitor, a NPY analogue, a NPY-1 agonist and a NPY-5 agonist for a time sufficient that it results in a decrease in the desire for consumption of food.

2. A method according to claim 1 wherein the composition is an NPY-1 agonist.

3. A method according to claim 1 wherein the composition is an NPY-5 agonist.

4. A method according to claim 1 wherein the composition is NPY.

5. A method according to claim 4 wherein the NPY is administered by administering to the patient a composition which causes increased production or release of NPY.

6. A method according to claim 4 wherein the NPY is administered by administering to the mammal a composition which causes decreased degradation of NPY.

7. A method for treating an eating related disease or condition in a human in need of such treatment comprising:
a) administering to the human a therapeutically effective amount of a pharmaceutically acceptable composition from the group comprising NPY, a NPY stimulant, a NPY degradation inhibitor, a NPY analogue, a NPY-1 agonist and a NPY-5 agonist;
b) continually administering the composition from a) while monitoring the effect of the composition and observing when appetite enhancement ends; and
c) continuing to administer the composition past the end of appetite enhancement for a desired period of time.

8. A method according to claim 6 wherein the composition is administered orally.

9. A method according to claim 7 wherein the composition is administered by a depot formulation.

10. A method according to claim 6 wherein the composition is administered with an additional appetite suppressant.

11. A method according to claim 7 wherein the desired period of time is as long as the disease or condition exists.
